(19) United States
(12) Patent Application Publication
(10) Pub. No.: US 2018/0162870 A1
(43) Pub. Date: Jun. 14, 2018

(54) Process for the Preparation of Darunavir

(71) Applicant: Amneal Pharmaceuticals Company GmbH, Steinhausen (CH)

(72) Inventors: Virendra Kumar Agarwal, Ahmedabad (IN); Lalit Keshav Katariya, Ahmedabad (IN); Ashish Rameshchandra Upadhyay, Dist. Banswara (IN); Ranjit Ravatbhai Pada, Dist. Bhavnagar (IN); Renish Mansukhlal Ghetiya, Dist: Rajkot (IN); Sabirhusen Ismalbhai Tuvar, Dist: Rajkot (IN)

(21) Appl. No.: 15/577,919

(22) PCT Filed: Jun. 3, 2016
(86) PCT No.: PCT/EP2016/062728

§ 371 Date: Nov. 29, 2017

(30) Foreign Application Priority Data
Jun. 5, 2015 (IN) 2174/MUM/2015

(51) Int. Cl. C07D 493/04; C07C 311/44; C07C 303/40

(52) U.S. Cl. CPC C07D 493/04; C07C 303/40; C07C 311/44

(57) ABSTRACT

The present invention provides a cost effective and industrially feasible process for preparation of Darunavir (I). Also described is the novel salt of intermediate 4-Amino-N-((2R,3S)-3-amino-2-hydroxy-4-phenylbutyl)-N-isobutylbenzene sulfonamide acid salt (VIII) and its use in the preparation of Darunavir. Formula (I) and (VII).

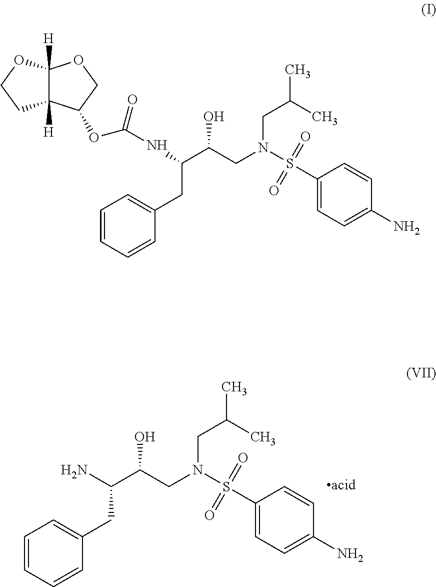 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1 ; CC(C)CN(C[C@@H](O)[C@@H](N)Cc1ccccc1)S(=O)(=O)c1ccc(N)cc1]

### FIELD OF INVENTION

The present invention relates to an improved process for preparation of Darunavir (I) or pharmaceutically acceptable salts thereof. The present invention also relates to the novel salt of intermediate and its use in the preparation of Darunavir.

### BACKGROUND OF INVENTION

Darunavir is potent HIV protease inhibitor that belongs to the class of hydroxyethyl amino sulfonamides. Darunavir is known by chemical name [(1S,2R)-3-[[(4-aminophenyl)sulfonyl](2-methylpropyl)amino]-2-hydroxy-1-(phenylmethyl)propyl]-carbamic acid (3R,3aS,6aR) hexahydrofuro[2,3-b]furan-3-yl ester. It is marketed in its monoethanolate form. Darunavir ethanolate is marketed in the United States of America by Tibotec Pharmaceuticals under the trade name Prezista®. Darunavir ethanolate is represented by following structure.

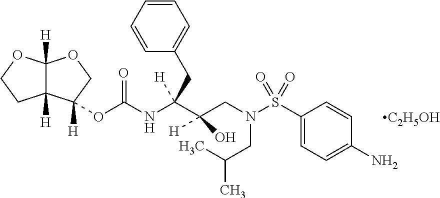 [SMILES: "·C2H5OH" ; CC(C)CN(C[C@@H](O)[C@H](Cc1ccccc1)NC(=O)O[C@H]1CO[C@H]2OCC[C@H]21)S(=O)(=O)c1ccc(N)cc1]

Darunavir is co-administered with ritonavir, and other antiretroviral agents. It is indicated for the treatment of human immunodeficiency virus (HIV-1) infection. It is also used with Cobicistat in a fixed dosage form.

Darunavir is generically disclosed in U.S. Pat. No. 5,843,946 and specifically disclosed in U.S. Pat. No. 6,248,775.

The process is described in Journal of Medicinal Chemistry, 2005, 48(6), 1813-1822 and U.S. Pat. No. 7,772,411 which involves condensation of diamino compound (VIII) with furanyl derivative (IXa) as depicted in scheme 1 below.

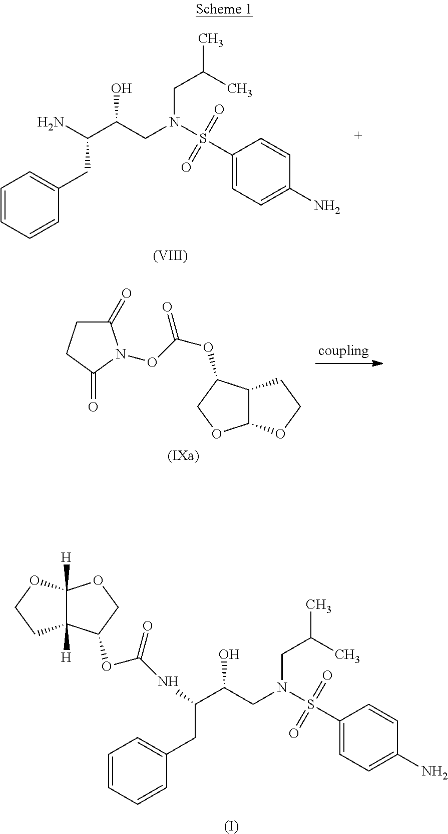 [SMILES: CC(C)CN(C[C@@H](O)[C@@H](N)Cc1ccccc1)S(=O)(=O)c1ccc(N)cc1 ; "(VIII)" ; O=C(O[C@H]1CO[C@H]2OCC[C@H]21)ON1C(=O)CCC1=O ; CC(C)CN(C[C@@H](O)[C@H](Cc1ccccc1)NC(=O)O[C@H]1CO[C@H]2OCC[C@H]21)S(=O)(=O)c1ccc(N)cc1 ; "(I)"]

2-R-hydroxy-3-[[(4-aminophenyl)sulfonyl](2-methylpropyl)amino]-1S-(phenylmethyl) propylamine (VIII) is reacted with (3R, 3aS, 6aR)-hexahydrofuro[2,3-b]furan-3-ol derivative (IXa) to give Darunavir. This process requires column chromatography to purify Darunavir which is not suitable for industrial scale process.

U.S. Pat. No. 7,772,411 discloses a process for the preparation as depicted in scheme 2

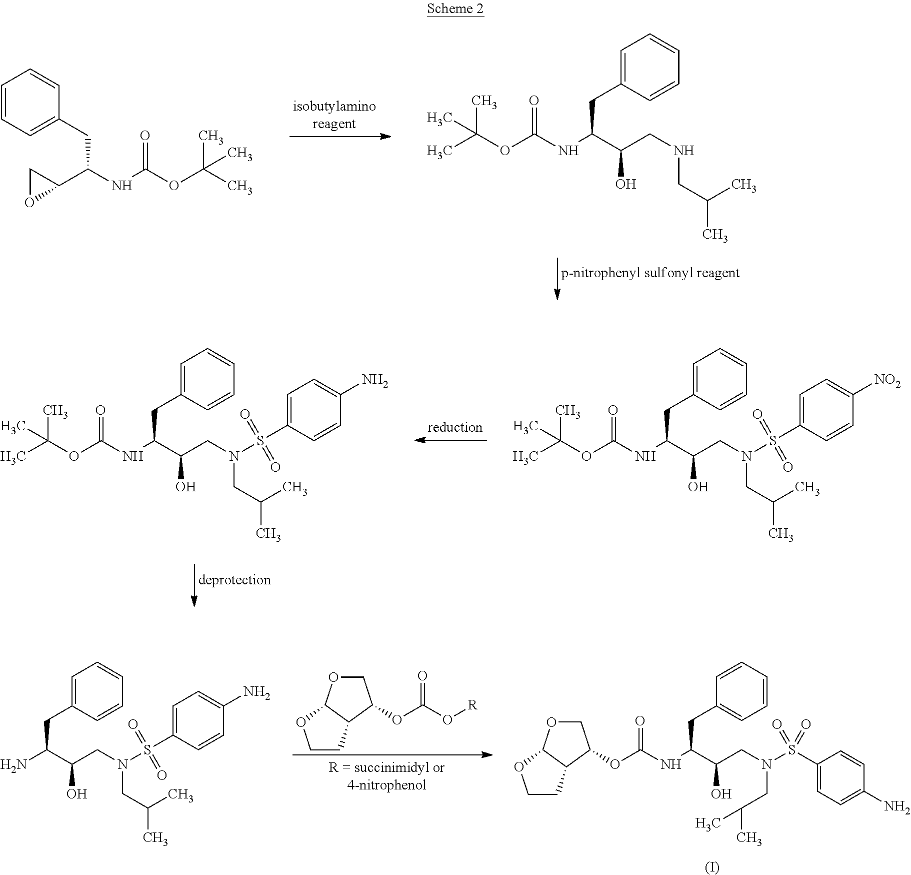

In the above process, nitro intermediate is required to reduce to amine which is done e by hydrogenation in presence of noble metal catalyst such as Pd or Pt in autoclave under hydrogen pressure. It requires catalyst such as Pd and Pt which tend to be high-priced. Further, such catalyst requires special treatment as they are sensitive and hazardous. The obtained amine requires separate deprotection step to remove t-butyloxy carbonyl (BOC) group, thereby increasing overall process steps, which in turn increase cost and reduce productivity.

There is an unmet need for a cost-effective and industrially feasible process for the preparation of Darunavir.

The present invention provides new approach for the preparation of Darunavir which involves acid addition (salt of amino intermediate (VII). The present invention also includes a process in which N-protected compound (V) is used as a starting material to synthesize compound (VI), which is more stable than respective amino compound prepared by reduction of nitro intermediate as shown in scheme 2. Further, deprotection of R₁ and R₂ groups takes place in a single step along with salt formation when treated with an acid. The acid addition salt (VII) is optionally isolated and purified to remove undesirable impurities, which otherwise remains in the final product, if proceed without the formation of an acid addition salt. This process eliminates the use of column chromatography to get pure Darunavir of pharmaceutical grade.

### SUMMARY OF THE INVENTION

The present invention relates to Darunavir, which is prepared in high purity and high yield.

In one aspect, the present invention provides a process for the preparation of Darunavir (I)

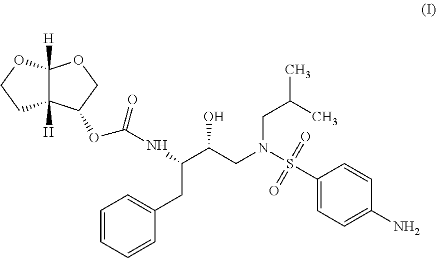 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]

which comprises:
a) aminating a compound of formula II with isobutyl amine (III)

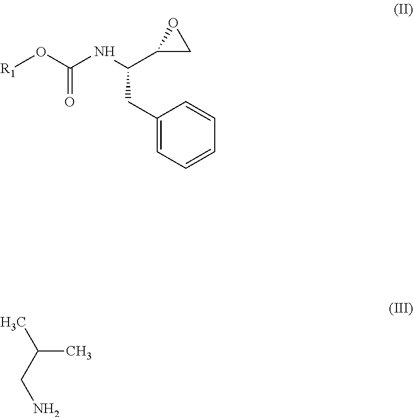

to give a compound of formula IV

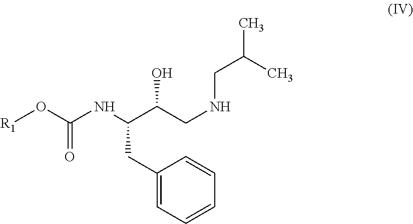

wherein R₁ is methyl, ethyl, t-butyl or benzyl;
b) reacting the compound of formula IV with N-protected 4-aminobenzenesulfonyl halide (V) to give a compound of formula VI

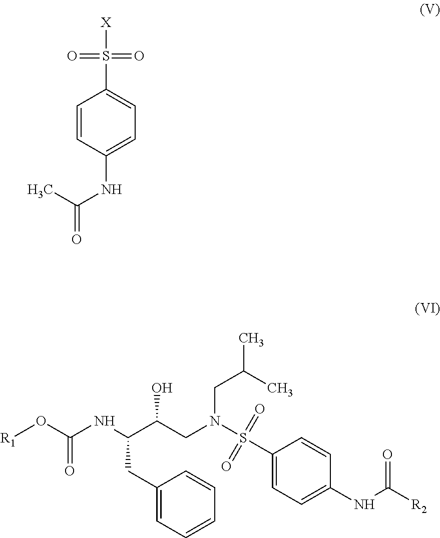

wherein X represents Cl, Br, I or F
wherein R₂ is H, methyl, trifluoromethyl or phenyl;
c) reacting the compound of formula VI with acid to give a compound of formula VII

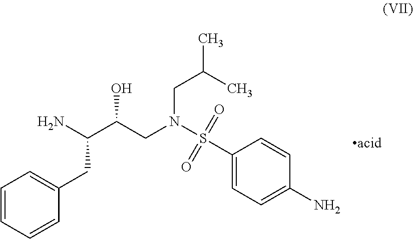 [SMILES: CC(C)CN(C[C@@H](O)[C@@H](N)Cc1ccccc1)S(=O)(=O)c1ccc(N)cc1]

wherein acid is selected from sulfuric acid, hydrobromic acid, nitric acid, methanesulfonic acid or phosphoric acid;
d) reacting the compound of formula VII with base to give 4-Amino-N-((2R,3S)-3-amino-2-hydroxy-4-phenylbutyl)-N-isobutylbenzene sulfonamide (VIII); and

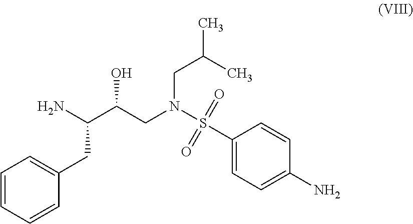 [SMILES: CC(C)CN(C[C@@H](O)[C@@H](N)Cc1ccccc1)S(=O)(=O)c1ccc(N)cc1]

e) reacting the compound of formula VIII with compound of formula IX to give Darunavir (I)

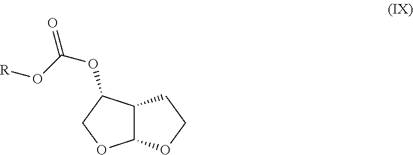

wherein R represents succinimidyl, 4-nitrophenyl, or imidazolyl.

In one aspect, the present invention may provide sulphate salt of 4-Amino-N-((2R,3S)-3-amino-2-hydroxy-4-phenylbutyl)-N-isobutylbenzene sulfonamide (VII).

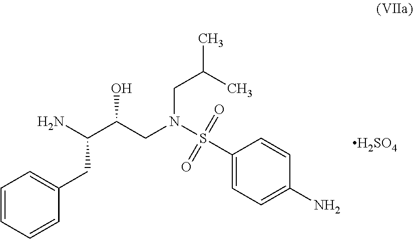 [SMILES: CC(C)CN(C[C@@H](O)[C@@H](N)Cc1ccccc1)S(=O)(=O)c1ccc(N)cc1 ; "·H2SO4"]

In another aspect, the present invention may provide a process for the preparation of 4-Amino-N-((2R,3S)-3-amino-2-hydroxy-4-phenylbutyl)-N-isobutylbenzene sulfonamide sulphate salt (VIIa) which comprises of a reaction of (1S,2R)-1-benzyl-2-hydroxy-3-[isobutyl-(4-acetylaminobenzenesulfonyl amino) propyl]carbamic acid tert-butyl ester (VI) with sulphuric acid to give 4-Amino-N-((2R,3S)-3-amino-2-hydroxy-4-phenylbutyl)-N-isobutylbenzene sulfonamide sulphate salt (VIIa)

The present invention may also provide a process for the preparation of Darunavir, which comprises the use of 4-Amino-N-((2R,3S)-3-amino-2-hydroxy-4-phenylbutyl)-N-isobutylbenzene sulfonamide sulphate salt (VIIa) as an intermediate.

In another aspect, the present invention may provide a process for the preparation of (1S,2R)-1-benzyl-2-hydroxy-3-[isobutyl-(4-acetylaminobenzenesulfonyl amino) propyl]carbamic acid tert-butyl ester (VI) which comprises of a reaction of (1S,2R)-(1-Benzyl-2-hydroxy-3-(isobutyl-amino)propyl)carbamic acid tert-butylester (IV) with 4-Acetylamino-benzenesulfonyl chloride (V).

### DETAIL DESCRIPTION OF INVENTION

All percentages and ratios used herein are by weight of the total composition and all measurements made are at about 25° C. and about normal pressure, unless the context requires otherwise. All temperatures are in degree Celsius unless specified otherwise. As used herein, “comprising” or “comprises” (open-ended) means the element or elements recited, or their equivalents in structure or function, plus any other element or elements which are not recited. The terms “having” and “including” are also to be construed as open-ended. All ranges recited herein include the endpoints, including those that recite a range “between” two values. Whether so indicated or not, all values recited herein are approximate as defined by the circumstances, including the degree of expected experimental error, technique error, and instrument error for a given technique used to measure a value. Unless defined otherwise, technical and scientific terms used herein have the same meaning as is commonly understood by one of skill in the art.

The term “about” when used in the present invention preceding a number and referring to it, is meant to designate any value which lies within the range of ±10%, preferably within a range of ±5%, more preferably within a range of ±2%, still more preferably within a range of ±1% of its value. For example “about 10” should be construed as meaning within the range of 9 to 11, preferably within the range of 9.5 to 10.5, more preferably within the range of 9.8 to 10.2, and still more preferably within the range of 9.9 to 10.1.

Unless specified otherwise, the word “pure” as used herein means that the material is at least about 99% pure. In general, this refers to purity with regard to unwanted residual solvents, reaction by-products, impurities, and unreacted starting materials. “Substantially pure” as used herein means at least about 98% pure and, likewise, “essentially pure” as used herein means at least about 95% pure.

“Substantially free of one or more of its corresponding impurities” as used herein, unless otherwise defined refers to the compound that contains at least less than about 2%, or less than about 1%, or less than about 0.5%, or less than about 0.3%, or less than about 0.2%, or less than about 0.1%, or less than about 0.05%, or less than about 0.03%, or less than about 0.01%, by weight, of each individual.

“Room temperature” as used herein refers to the temperature as understood by one skilled in the art to be in range of about 20° C. to about 30° C. A process or step may be referred to herein as being carried out “overnight”. This refers to a time interval, e.g., for the process or step, that spans the time during the night, when that process or step may not be actively observed. This time interval is from about 8 to about 18 hours, typically about 16 hours. Unless otherwise indicated, the solid state forms of the present invention may be dried. Drying may be carried out using methods and equipments as known by one skilled in the art such in a tray dryer, vacuum oven, Buchi® Rotavapor®, air oven, fluidized bed dryer, spin flash dryer, flash dryer, cone dryer, agitated nutsche filter cum dryer, nauta dryer or the like or any other suitable dryer.

The drying may be carried out at a temperature between about −20° C. to about 150° C. The drying may be carried out under reduced atmospheric pressure, that is, less than standard atmospheric pressure or at atmospheric pressure or any other suitable pressure. The drying may take place over a period of about 30 minutes to about 12 hours. The dried product may be subjected to techniques such as sieving to get rid of lumps before and/or after drying. The dried product may be optionally milled to get a desired particle size. Milling or micronization may be performed before drying and/or after the completion of drying of the product. Techniques that may be used for particle size reduction include, without limitation, ball, roller and hammer mills, and jet mills.

The term “amination” and its variation as used herein refer to a process in which a primary amine, isobutylamine, is introduced into the organic molecule of formula (II). Amination of compound of formula (II) may be accomplished in several ways available in the literature, for example as described in WO95/06030, which is incorporated herein by reference. Amination of epoxides is described for instance in March, Advanced Organic Chemistry 368-69 (3rd Ed. 1985) and McManus et al, Synth. Comm. 177 (1973), which are incorporated herein by reference.

The amination agent, isobutylamine, may also function as a solvent, in which case, an excess of isobutylamine will be added. In some embodiments, the amination process is performed in the presence of one or more solvents other than isobutylamine.

Examples of solvents that may be suitable for use in the present invention include protic, non-protic and dipolar aprotic organic solvents such as, for example, those wherein the solvent is an alcohol, such as methanol, ethanol, isopropanol, n-butanol, t-butanol, and the like; ketones such as acetone; ethers such as diethyl ether, tetrahydrofuran, dioxane and the like; esters such as ethyl acetate, aminos such as triethylamine; amides such as N,N-dimethylformamide, or dimethylacetamide; chlorinated solvents such as dichloromethane and other solvents such as toluene, dimethyl sulfoxide, acetonitrile, and mixtures thereof.

The present invention provides a process for the preparation of Darunavir (I)

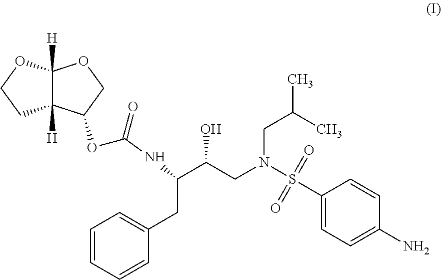 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]

which comprises:
a) aminating a compound of formula II with isobutyl amine (III)

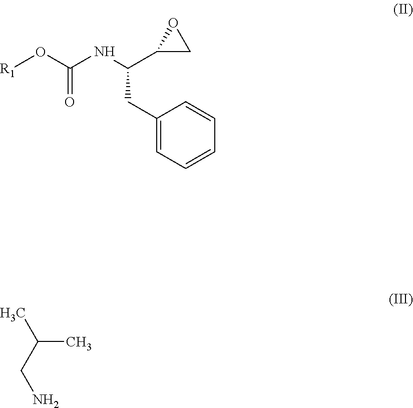

to give a compound of formula IV

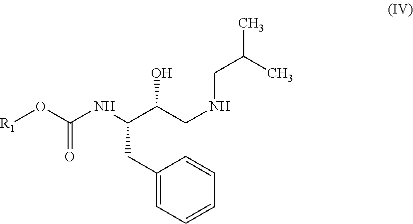

wherein R₁ is methyl, ethyl, t-butyl or benzyl;
b) reacting the compound of formula IV with N-protected 4-aminobenzenesulfonyl halide (V) to give a compound of formula VI

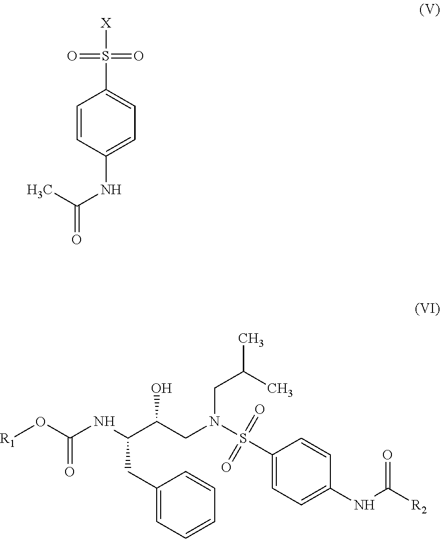

wherein X represents Cl, Br, I or F
wherein R₂ is H, methyl, trifluoromethyl or phenyl;
c) reacting the compound of formula VI with acid to give a compound of formula VII

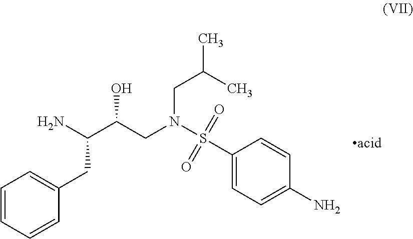 [SMILES: CC(C)CN(C[C@@H](O)[C@@H](N)Cc1ccccc1)S(=O)(=O)c1ccc(N)cc1]

wherein acid is selected from sulfuric acid, hydrobromic acid, nitric acid, methanesulfonic acid or phosphoric acid;
d) reacting the compound of formula VII with base to give 4-Amino-N-((2R,3S)-3-amino-2-hydroxy-4-phenylbutyl)-N-isobutylbenzene sulfonamide (VIII); and

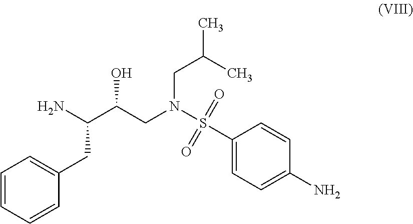 [SMILES: CC(C)CN(C[C@@H](O)[C@@H](N)Cc1ccccc1)S(=O)(=O)c1ccc(N)cc1]

e) reacting the compound of formula VIII with compound of formula IX to give Darunavir (I)

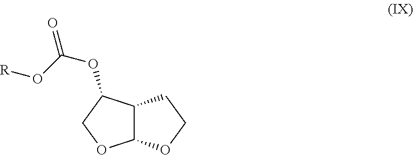

wherein R represents succinimidyl, 4-nitrophenyl, or imidazolyl.

In step a), (2S,3S)-1,2-Epoxy-3-(N-protected-amino)-4-phenylbutane (II) is aminated with isobutyl amine (III) to obtain compound of formula IV.

The reaction in the present invention can be conducted over a range of temperatures, e.g., from about −20° C. to about 200° C., but is preferably, conducted at a temperature at which the solvent refluxes, i.e. between 40° C. to about 100° C., more preferably between 60° C. to about 90° C.

The ratio of equivalents between the compound of formula (II) and the amination agent may range from 1:1 to about 1:99, respectively. Preferably, the ratio of equivalents between the compound of formula (II) and the amination agent is from 1:5 to about 1:20, more preferably the ratio is from 1:10 to about 1:15.

In step b), reaction of the compound of formula IV with N-protected 4-aminobenzenesulfonyl halide (V) to obtain a compound of formula VI can be performed in presence of a base and a solvent. The base used may be organic or inorganic base. Examples of organic base that may be used in the present invention include triethylamine (TEA), diethylmethylamine, pyridine, 1,8-diazabicyclo[5.4.0]undec-7-ene (DBU), N methyl 2-pyrrolidone (NMP), diisopropyl ethylamine (DIPEA), N,N-dimethyl amino pyridine (DMAP), imidazole and the like. Inorganic base used in the present invention may include but are not limited to carbonate, bicarbonate, hydride of alkali and alkaline earth metals such as sodium carbonate, sodium bicarbonate, potassium carbonate, potassium bicarbonate, lithium carbonate, cesium carbonate, sodium hydride and the like. The solvent that may be used are organic solvent. Examples of organic solvents that may be used in the present invention include N,N-Dimethylacetamide (DMAc), N.N dimethyl formamide (DMF), NMP, methylene dichloride (MDC), ethylene dichloride (EDC), toluene, ethylacetate (EtOAc), isopropylacetate (iPrOAc), acetone, methylethyl ketone (MEK), tetrahydrofuran (THF), acetonitrile (ACN) and the like. Preferably, the based used in step (b) is triethylamine (TEA).

In the present invention, the ratio of equivalents, calculated from compounds of formula IV, and the compound of formula V range from 1:1 to about 1:3, respectively. Preferably, the ratio of equivalents between the compounds of formula IV and the compound of formula V range from 1:1 to about 1:2, more preferably the ratio can be about 1:1.15.

In step c), reacting the compound of formula VI with acid to give a compound of formula VII. The acid used can be selected from sulfuric acid, hydrobromic acid, nitric acid, methanesulfonic acid or phosphoric acid. Optionally, step (c) is performed in the presence of an organic solvent. The organic solvent used in the present invention maybe a polar organic solvent, which includes both protic and aprotic solvents. Examples of suitable polar solvents include C₃-C₁₀ aliphatic ketones (e.g., acetone, methyl ter-butyl ketone, etc.), C₁-C₆ chlorinated hydrocarbons (e.g., dichloromethane), C₁-C₆ aliphatic alcohols (e.g., methanol, ethanol, propanol, isopropanol), C₃-C₁₀ aliphatic esters (e.g., ethyl acetate), C₂-C₅ aliphatic nitriles (e.g., acetonitrile), and ethers, including cyclic ethers (e.g., di-isopropyl ether, tetrahydrofuran), as well as mixtures thereof.

In some embodiments of the present invention, the solvent maybe stirred at 0° C. to a reflux temperature of the solvent used. The acid may be added in a quantity equimolar to the amount of the compound of formula VI which may be present in an amount of about 1.0 to 1.2 eq., more preferably 1.0 to 1.05 eq.

In one of the embodiments, the acid addition salt (VII) is isolated and purified to remove undesirable impurities, which otherwise remain in the final product. The obtained acid addition salt (VII) can be further recrystallized in order to obtain higher purity. The recrystallization may be performed using procedures generally known in the art.

In some embodiments, the salt form may be isolated, for example, by concentrating the reaction mixture, or alternatively, by cooling the reaction mixture (with or without concentrating the mixture first) and isolating the resulting precipitate by filtration. As used herein, the term “isolated” does not require absolute purity, but rather is intended as a relative term. Thus, for example, an isolated compound can be one in which the subject compound is at a higher concentration than in the environment from which it was removed.

In a preferred embodiment, (1S,2R)-1-benzyl-2-hydroxy-3-[isobutyl-(4-acetylamino benzenesulfonylamino) propyl]carbamic acid tert-butyl ester (VI) may be reacted with aq. sulphuric acid in alcohol such as isopropyl alcohol, methanol, ethanol, propanol and the like. The reaction proceeds between ambient temperature to reflux temperature at about 84° C. The reaction mixture is filtered and treated in alcohol to give 4-Amino-N-((2R,3S)-3-amino-2-hydroxy-4-phenylbutyl)-N-isobutylbenzene sulfonamide sulphate salt (VIIa).

In step d), the compound of formula VII is reacted with base to give 4-Amino-N-((2R,3S)-3-amino-2-hydroxy-4-phenylbutyl)-N-isobutylbenzene sulfonamide (VIII). The reaction may be carried out in presence of water or alcohol; such as methanol, ethanol, isopropanol, n-butanol, t-butanol, and the like; ketones such as acetone; ethers such as diethyl ether, tetrahydrofuran, dioxane and the like; esters such as ethyl acetate, aminos such as triethylamine; amides such as N,N-dimethylformamide, or dimethylacetamide; chlorinated solvents such as dichloromethane and other solvents such as toluene, dimethyl sulfoxide, acetonitrile, and mixtures thereof. The base are inorganic base such as aq. potassium carbonate, sodium carbonate, sodium or potassium bicarbonate, lithium carbonate, cesium carbonate or organic base such as TEA, diethylmethylamine, pyridine, DBU, NMP, DIPEA, DMAP, imidazole. The amount of the base can be in the range of 1 to about 10 equivalents, preferably in range of 1 to about 5 equivalents, based on 1 equivalent of the compound of formula VII.

In a preferred embodiment, a mixture of 4-Amino-N-((2R,3S)-3-amino-2-hydroxy-4-phenylbutyl)-N-isobutylbenzene sulfonamide sulphate salt (VIIa) in water is heated with aq. solution of base to give 4-Amino-N-((2R,3S)-3-amino-2-hydroxy-4-phenylbutyl)-N-isobutylbenzene sulphonamide (VIII).

In step e), the compound of formula VIII is reacted with compound of formula IX to give Darunavir (I). Compound of formula IX is activated derivative of (3R,3aS,6aR)-hexahydrofuro[2,3-b]furan-3-ol prepared using bis-(4-nitrophenyl)carbonate, disuccinimidyl carbonate (DSC), carbonyl diimidazole (CDI).

Reaction of compound of formula IX with compound of formula VII can be performed in the presence of suitable solvent and base. Suitable solvents include protic, non-protic and dipolar aprotic organic solvents such as, for example, those wherein the solvent is an alcohol, such as methanol, ethanol, isopropanol, n-butanol, t-butanol, and the like; ketones such as acetone; ethers such as diethyl ether, tetrahydrofuran, dioxane and the like; esters such as ethyl acetate, aminos such as triethylamine; amides such as N,N-dimethylformamide, or dimethylacetamide; chlorinated solvents such as dichloromethane and other solvents such as toluene, dimethyl sulfoxide, acetonitrile, and mixtures thereof. The base used may be organic or inorganic base. Orgainc base are triethylamine (TEA), diethylmethylamine, pyridine, 1,8-diazabicyclo[5.4.0]undec-7-ene (DBU), N methyl 2-pyrrolidone (NMP), diisopropyl ethylamine (DIPEA), N,N-dimethyl amino pyridine (DMAP), imidazole and the like. Inorganic base are carbonate, bicarbonate, hydride of alkali and alkaline earth metals such as sodium carbonate, sodium bicarbonate, potassium carbonate, potassium bicarbonate, lithium carbonate, cesium carbonate, sodium hydride and the like.

During the reaction of the compound of formula VIII with compound of formula IX, compound of formula IX can be present at a concentration between 1% and 15% (W/W), preferably at a concentration between 5% and 12% (W/W), more preferably at a concentration between 8% and 12% (W/W). The reaction can be suitably carried out at a temperature between −70° C. and 40° C., preferably between −10° C. and 20° C.

In preferred embodiment, 4-Amino-N-((2R,3S)-3-amino-2-hydroxy-4-phenylbutyl)-N-isobutylbenzene sulphonamide (VIII) is stirred with base and a solvent followed by addition of [(3R,3aS,6aR)-Hydroxyhexahydrofuro[2,3-b]furanyl succinimidyl Carbonate (IX)(R=succinimidyl).

In preferred embodiment, Darunavir obtained by process of the present invention is in amorphous form or a crystalline form.

Darunavir (I) may be further solvated with alcohols such as ethanol, methanol, being the ethanolate solvate form preferred. Solvation of Darunavir is described in PCT/EP03/50176 (Tibotec N.V.), which is incorporated herein by reference.

In some embodiments, processes of the present application provide Darunavir having purity by HPLC which may be pure, substantially pure or essentially pure.

In other embodiments, processes of the present application provide Darunavir substantially free of one or more of impurities.

In some embodiments, processes of the present application provide Darunavir substantially free of bis-impurity of following structure:

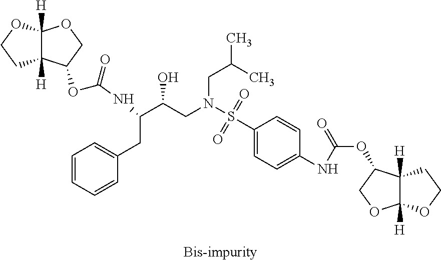 [SMILES: CC(C)CN(C[C@@H](O)[C@H](Cc1ccccc1)NC(=O)O[C@H]1CO[C@H]2OCC[C@H]21)S(=O)(=O)c1ccc(NC(=O)O[C@H]2CO[C@H]3OCC[C@H]32)cc1]

In a preferred embodiment, the process of present invention can be as depicted scheme 3.

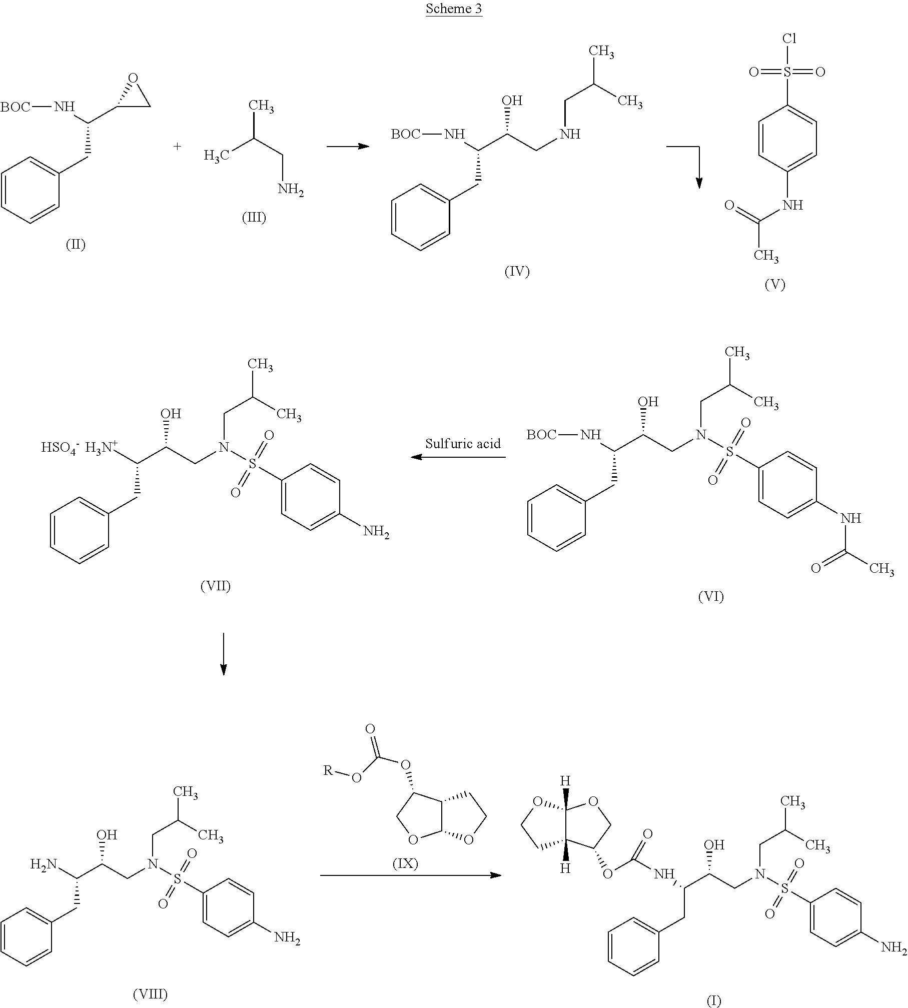

The following examples are given for the purpose of illustrating the present invention and should not be considered as limiting on the scope of the invention.

### EXAMPLE 1

### Preparation of (1S,2R)-(1-Benzyl-2-hydroxy-3-(isobutyl-amino)propyl)carbamic acid tert-butylester (IV)

A mixture of (2S,3S)-1,2-Epoxy-3-(Boc-amino)-4-phenylbutane (II) (100 g) and isobutyl amine (III) (277.9 g) was heated at 65-75° C. for 3 hours. After completion of the reaction, Isobutyl amine was distilled out completely from reaction mixture at 75-85° C. The residue was cooled below 50° C. and Methanol (200 ml) was added. The mixture was heated at 60-70° C. to make a clear solution. The solution was cooled at 25-35° C. and added to water (1000 ml) and stirred for 2 hours. The product was filtered and washed with water (2×50 ml) to get wet cake. The wet cake was re-slurried in water (1500 ml), stirred for 2 hours and filtered. The wet cake was washed with water (2×50 ml). Solid was dried in air tray dryer for 2 hours at 25-35° C. and then at 65-75° C. for 12 hours to give the title product (124.0 g)

Yield: 97.63%

### EXAMPLE 2

### Preparation of (1S,2R)-1-benzyl-2-hydroxy-3-[isobutyl-(4-acetylaminobenzenesulfonyl amino) propyl]carbamic acid tert-butyl ester (VI)

N-Acetyl Sulphanilyl chloride (72.9 g) (V) was added at 05-15° C. to pre-cooled mix of (1S,2R)-(1-Benzyl-2-hydroxy-3-(isobutyl-amino)propyl)carbamic acid tert-butylester (IV) (100 g) in N,N-Dimethylacetamide (500 ml). Triethyl amine (36.1 g) was added to the reaction mixture below 30° C. and stirred for 2 hours at 25-35° C. Water (100 ml) was added to the reaction mixture at 25-35° C. This mixture was added to water (2000 ml) at 25-35° C. under stirring and stirred for 2 hours. The reaction mixture was filtered and solid was washed with water (2×100 ml). The wet cake is reslurried in to water (1500 ml) and stirred for 2 hours. The solid was filtered and washed with water (2×100 ml). Solid was dried in air tray dryer for 2 hours at 25-35° C. and then at 65-75° C. for 12 hours to give the title product (144.0 g)

Yield: 90.56%

₁H NMR (DMSO-d₆) δppm: 0.78-0.85 (6H, dd), 1.11-1.25 (9H, m), 1.93-2.00 (1H, m), 2.08 (3H, s), 2.48-2.54 (1H, m), 2.74-2.84 (2H, m), 2.96-3.03 (2H, m), 3.31-3.35 (1H, m), 3.46-3.51 (1H, m), 3.56-3.60 (1H, m), 4.96-4.97 (1H, d), 6.68-6.70 (1H, d), 7.12-7.25 (5H, m), 7.69-7.78 (4H, m), 10.32 (1H, s)

Mass: 534.2 (M+H)⁺

Purity: 96.78%

### EXAMPLE 3

### (a) Preparation of 4-Amino-N-((2R,3S)-3-amino-2-hydroxy-4-phenylbutyl)-N-isobutylbenzenesulfonamide sulphate salt (VII)

(1S,2R)-1-benzyl-2-hydroxy-3-[isobutyl-(4-acetylaminobenzenesulfonylamino)propyl] carbamic acid tert-butyl ester (100 g) (VI) was added in to Isopropyl alcohol (1200 ml) at 25-35° C. Aqueous sulphuric acid solution (131.2 g Sulphuric acid in 150 ml water) was added to reaction mixture at 25-35° C. Reaction mixture was heated at 80-90° C. and stirred for 12 hours. The reaction mixture was cooled at 25-35° C. for 2 hours and then at 0-10° C. for 2 hours. The solid is filtered and washed with Isopropyl alcohol (2×50 ml). The solid was added to mixture of isopropyl alcohol (1000 ml) and water (100 ml) at 25-35° C. and then heated at 65-75° C. for 30 minutes. The reaction mixture was cooled to 25-35° C. for 2 hours and then to 0-10° C. for 2 hours and filtered. The solid obtained was washed with isopropyl alcohol (2×50 ml) to give 4-Amino-N-((2R,3S)-3-amino-2-hydroxy-4-phenylbutyl)-N-isobutylbenzene sulfonamide sulphate salt. (75.0 g).

Sulfate content (On Anhydrous basis): 21.51%.

¹H NMR (DMSO) δ: 0.77-0.85 (6H, dd), 1.83-1.90 (1H, m), 2.57-2.62 (1H, m), 2.68-2.77 (2H, m), 2.84-2.90 (1H, m), 3.04-3.09 (1H, m), 3.30-3.35 (4H, m), 3.49 (2H, broad), 3.99-4.00 (1H, m), 5.59-5.60 (1H, d), 6.05 (2H, s), 6.61-6.63 (1H, d), 7.27-7.31 (2H, m), 7.36-7.41 (6H, m), 7.83 (3H, broad)

### (b) Preparation of 4-Amino-N-((2R,3S)-3-amino-2-hydroxy-4-phenylbutyl)-N-isobutylbenzenesulfonamide (VIII)

A mixture of Amino-N-((2R,3S)-3-amino-2-hydroxy-4-phenylbutyl)-N-isobutylbenzene sulfonamide sulphate salt (VII) as obtained above in water (700 ml) was heated at 50-60° C. Potassium carbonate Solution in water (38.8 g Potassium carbonate in water 200 ml) was added to the reaction mixture at 50-60° C. within 1 hour. Reaction mixture was stirred for 2 hours at 50-60° C. Reaction mixture was cooled to 25-35° C. and stirred for 2 hours. The product was filtered and washed with water (2×100 ml). The obtained solid was dried in air tray dryer for 2 hours at 25-35° C. and then at 65-75° C. for 12 hours to give the title product (56.0 g).

Yield=76.39%

¹H NMR (DMSO) δ: 0.78-0.83 (6H, dd), 1.21 (2H, s), 1.90-1.97 (1H, m), 2.30-2.36 (1H, m), 2.67-2.74 (1H, m), 2.78-2.91 (4H, m), 3.35-3.38 (1H, m), 3.49-3.50 (1H, m), 4.65-4.66 (1H, d), 5.97 (1H, s), 6.59-6.63 (2H, m), 7.16-7.23 (3H, m), 7.26-7.30 (2H, m), 7.39-7.42 (2H, m).

Mass: 392.1 (M+H)⁺

Purity: 99.96%

### EXAMPLE 4

### Preparation of [(1S,2R)-3-[[(4-aminophenyl)sulfonyl](2-methylpropyl) amino]-2-hydroxy-1(phenylmethyl)propyl]-carbamicacid(3R,3aS,6aR)-hexahydrofuro[2,3-b]furan-3-ylester (I) OR Darunavir

To a stirred mixture of potassium carbonate (28.2 g), isopropyl acetate (500 ml) and water (300 ml), 4-Amino-N-((2R,3S)-3-amino-2-hydroxy-4-phenylbutyl)-N-isobutylbenzene sulphonamide (100 g) was Charged at 25-35° C., stirred and cooled to 15-25° C. (3R,3aS,6aR)-Hydroxyhexahydrofuro[2,3-b]furanyl Succinimidyl Carbonate (69.3 g) was added to the reaction mixture at 15-25° C. and stirred for 4 hours. Stirring was stopped and settle layers for 30 minutes. Upper organic layer containing product was separated and washed with water (500 ml×2). Organic layer was added to the mixture of cyclohexane (1000 ml) and Methanol (100 ml) at 25-35° C., cooled to 0-10° C. and stirred for 2 hours. The solid was filtered and washed with cyclohexane (2×50 ml). Solid was dried in air tray dryer for 2 hours at 25-35° C. and then at 55-65° C. for 12 hours to give the title product (120.0 g).

Yield: 83.33%

¹H NMR (DMSO-d₆) δ: 0.79-0.87 (6H, dd), 1.19-1.24 (1H, m), 1.35-1.41 (1H, m), 1.93-1.96 (1H, m), 2.43-2.46 (1H, m), 2.59-2.66 (2H, m), 2.74-2.80 (1H, m), 2.92-3.06 (2H, m), 3.27-3.31 (1H, m), 3.52-3.64 (4H, m), 3.71-3.75 (1H, m), 3.85-3.89 (1H, m), 4.82-4.87 (1H, m), 5.00-5.02 (1H, d), 5.51-5.52 (1H, d), 5.99 (2H, s), 6.59-6.61 (2H, d), 7.12-7.16 (1H, m), 7.21-7.24 (4H, m), 7.27-7.30 (1H, m), 7.37-7.39 (2H, m)

Purity: 97.98%

Purification of Darunavir

[(1S,2R)-3-[[(4-aminophenyl)sulfonyl](2-methylpropyl) amino]-2-hydroxy-1-(phenyl methyl) propyl]-carbamicacid(3R,3aS,6aR)-hexahydrofuro[2,3-b]furan-3-ylester (Darunavir crude) was added to Methanol (300 ml) and heated at 55-65° C. for 30 minutes. The reaction mixture was cooled to 45-55° C. and filtered through hyflow bed. The bed was washed with Methanol (50 ml×2) and combined filtrate. The filtrate was heated at 55-65° C. for 30 minutes. The reaction mixture was cooled to 25-35° C. and stirred for 30 minutes and then to 0-10° C. and stirred for 2 hours. The mixture was filtered and solid was washed with chilled methanol (0-10° C.) (2×50 ml). Solid was dried in air tray dryer for 2 hours at 25-35° C. and then at 55-65° C. for 12 hours to give the title product (91 g).

Yield: 91.0%

¹H NMR (DMSO-d₆) δ: 0.79-0.87 (6H, dd), 1.19-1.24 (1H, m), 1.35-1.41 (1H, m), 1.93-1.96 (1H, m), 2.43-2.46 (1H, m), 2.59-2.66 (2H, m), 2.74-2.80 (1H, m), 2.92-3.06 (2H, m), 3.27-3.31 (1H, m), 3.52-3.64 (4H, m), 3.71-3.75 (1H, m), 3.85-3.89 (1H, m), 4.82-4.87 (1H, m), 5.00-5.02 (1H, d), 5.51-5.52 (1H, d), 5.99 (2H, s), 6.59-6.61 (2H, d), 7.12-7.16 (1H, m), 7.21-7.24 (4H, m), 7.27-7.30 (1H, m), 7.37-7.39 (2H, m)

Purity: 99.92%

1. A process for the preparation of Darunavir (I) 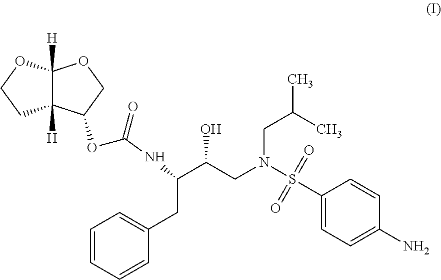 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]
which comprises:
a) aminating a compound of formula II with isobutyl amine (III) 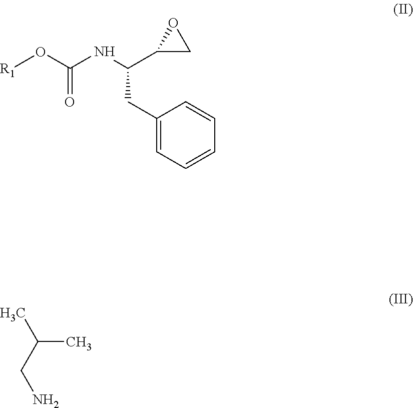
to give a compound of formula IV 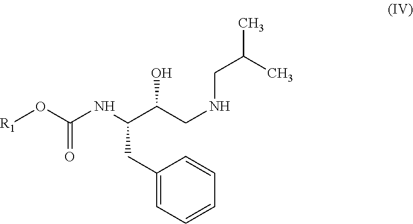
wherein R₁ is methyl, ethyl, t-butyl or benzyl;
b) reacting the compound of formula IV with N-protected 4-aminobenzenesulfonyl halide (V) to give a compound of formula VI 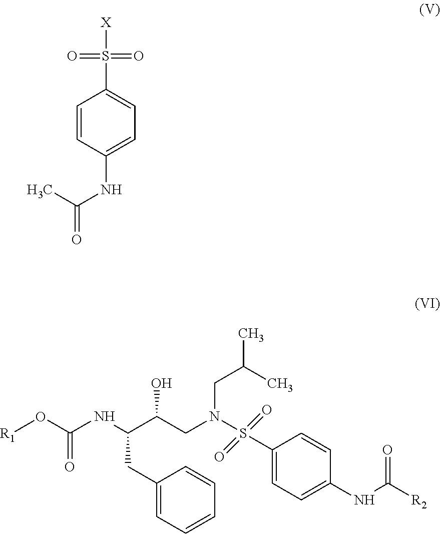
wherein X is Cl, Br, I or F,
wherein R₂ is H, methyl, trifluoromethyl or phenyl;
c) reacting the compound of formula VI with acid selected from sulfuric acid, hydrobromic acid, nitric acid, methanesulfonic acid or phosphoric acid to give a compound of formula VII 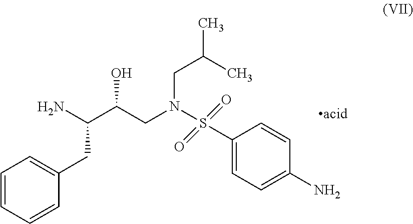 [SMILES: CC(C)CN(C[C@@H](O)[C@@H](N)Cc1ccccc1)S(=O)(=O)c1ccc(N)cc1]
d) reacting the compound of formula VII with base to give 4-Amino-N-((2R,3S)-3-amino-2-hydroxy-4-phenylbutyl)-N-isobutylbenzene sulfonamide (VIII); and 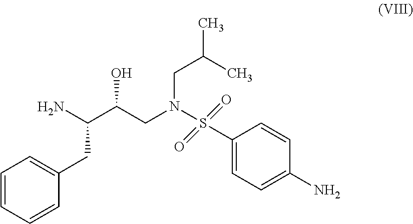 [SMILES: CC(C)CN(C[C@@H](O)[C@@H](N)Cc1ccccc1)S(=O)(=O)c1ccc(N)cc1]
e) reacting the compound of formula VIII with compound of formula IX to give Darunavir (I) 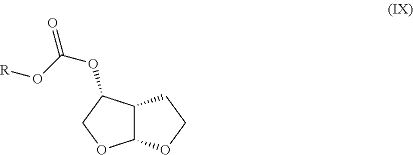
wherein R represents succinimidyl, 4-nitrophenyl, or imidazolyl.

2. The process according to claim 1, wherein step b) is carried out in presence of a base selected from organic base such as triethylamine (TEA), diethylmethylamine, pyridine, 1,8-diazabicyclo[5.4.0]undec-7-ene (DBU), N methyl 2-pyrrolidone (NMP), diisopropyl ethylamine (DIPEA),N,N-dimethyl amino pyridine (DMAP), carbonate bicarbonate or hydride of alkali and alkaline earth metals such as sodium carbonate, sodium bicarbonate, potassium carbonate, potassium bicarbonate, lithium carbonate, cesium carbonate, sodium hydride or mixtures thereof; and a solvent selected from N,N-Dimethylacetamide (DMAc), N.N dimethyl formamide (DMF), NMP, methylene dichloride (MDC), ethylene dichloride (EDC), toluene, ethylacetate (EtOAc), isopropylacetate (iPrOAc), acetone, methylethyl ketone (MEK), tetrahydrofuran (THF), acetonitrile (ACN) or mixture thereof.

3. The process according to claim 1, wherein step c) is carried out in presence of a solvent selected from C₃-C₁₀ aliphatic ketones such as acetone, methyl ter-butyl ketone, C₁-C₆ chlorinated hydrocarbons such as dichloromethane, C₁-C₆ aliphatic alcohols such as methanol, ethanol, propanol, isopropanol, C₃-C₁₀ aliphatic esters such as ethyl acetate, C₂-C₅ aliphatic nitriles such as acetonitrile, ethers such di-isopropyl ether, tetrahydrofuran, or mixture thereof.

4. The process according to claim 1, wherein the compound (VII) is either isolated or carried forward to next step in situ.

5. The process according to claim 1, wherein step d) base is inorganic base or organic base selected from potassium carbonate, sodium carbonate, sodium or potassium bicarbonate, lithium carbonate, cesium carbonate, TEA, diethylmethylamine, pyridine, DBU, NMP, DIPEA, DMAP, imidazole; and solvent is selected from water, methanol, ethanol, isopropanol, n-butanol, t-butanol, acetone, diethyl ether, tetrahydrofuran, dioxane, ethyl acetate, triethylamine, N,N-dimethylformamide, or dimethylacetamide, dichloromethane, toluene, dimethyl sulfoxide, acetonitrile or mixtures thereof.

6. The process according to claim 1, wherein step e) is carried out in presence of a base selected from organic or inorganic base such as triethylamine (TEA), diethylmethylamine, pyridine, 1,8-diazabicyclo[5.4.0]undec-7-ene (DBU), N methyl 2-pyrrolidone (NMP), diisopropyl ethylamine (DIPEA), N,N-dimethyl amino pyridine (DMAP), imidazole, carbonate, bicarbonate or hydride of alkali and alkaline earth metals such as sodium carbonate, sodium bicarbonate, potassium carbonate, potassium bicarbonate, lithium carbonate, cesium carbonate, sodium hydride or mixtures thereof; and solvent selected from methanol, ethanol, isopropanol, n-butanol, t-butanol, acetone, diethyl ether, tetrahydrofuran, dioxane, ethyl acetate, triethylamine, N,N-dimethylformamide, dimethylacetamide, dichloromethane, toluene, dimethyl sulfoxide, acetonitrile or mixtures thereof.

7. A compound of formula (VII) 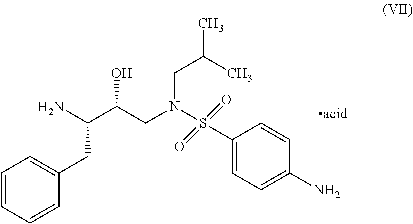 [SMILES: CC(C)CN(C[C@@H](O)[C@@H](N)Cc1ccccc1)S(=O)(=O)c1ccc(N)cc1]
wherein acid is selected from sulfuric acid, hydrobromic acid, nitric acid, methanesulfonic acid or phosphoric acid.

8. A process for preparation of Darunavir (I) 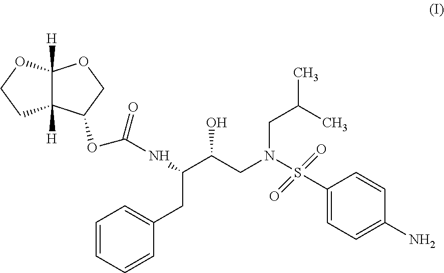 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]
which comprises reacting the compound of formula VI with acid selected from sulfuric acid, hydrobromic acid, nitric acid, methanesulfonic acid or phosphoric acid to give a compound of formula VII, 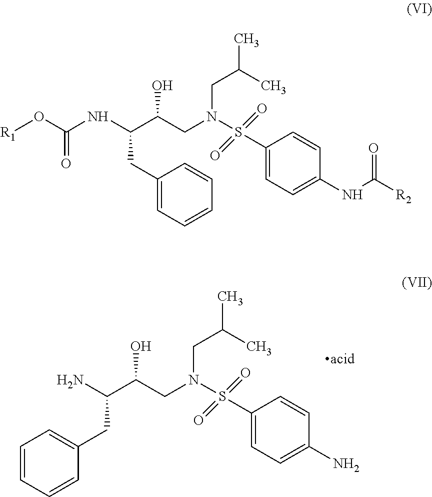
wherein R₁ is methyl, ethyl, t-butyl or benzyl; R₂ is H, methyl, trifluoromethyl or phenyl.

9. A process for preparing 4-Amino-N-((2R,3S)-3-amino-2-hydroxy-4-phenylbutyl)-N-isobutylbenzene sulfonamide (VIII) comprising:
a) reacting the compound of formula VI with acid selected from sulfuric acid, hydrobromic acid, nitric acid, methanesulfonic acid or phosphoric acid to give a compound of formula VII 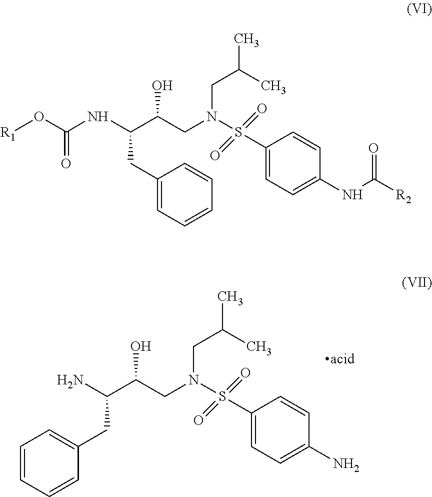
wherein R₁ is methyl, ethyl, t-butyl or benzyl; R₂ is H, methyl, trifluoromethyl or phenyl.
b) reacting the compound of formula VII with base to give 4-Amino-N-((2R,3S)-3-amino-2-hydroxy-4-phenylbutyl)-N-isobutylbenzene sulfonamide (VIII).

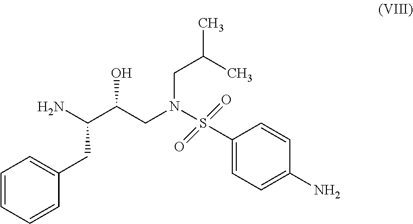 [SMILES: CC(C)CN(C[C@@H](O)[C@@H](N)Cc1ccccc1)S(=O)(=O)c1ccc(N)cc1]

10. The process according to claim 9, wherein base is inorganic base or organic base selected from potassium carbonate, sodium carbonate, sodium or potassium bicarbonate, lithium carbonate, cesium carbonate, TEA, diethylmethylamine, pyridine, DBU, NMP, DIPEA, DMAP, imidazole; and solvent is selected from water, methanol, ethanol, isopropanol, n-butanol, t-butanol, acetone, diethyl ether, tetrahydrofuran, dioxane, ethyl acetate, triethylamine, N,N-dimethylformamide, or dimethylacetamide, dichloromethane, toluene, dimethyl sulfoxide, acetonitrile or mixtures thereof.

11. A process for preparation of Darunavir (I): 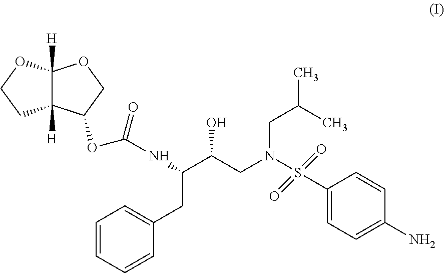 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]
which comprises,
a) reacting the compound of formula IV with N-protected 4-aminobenzenesulfonyl halide (V) to give a compound of formula VI 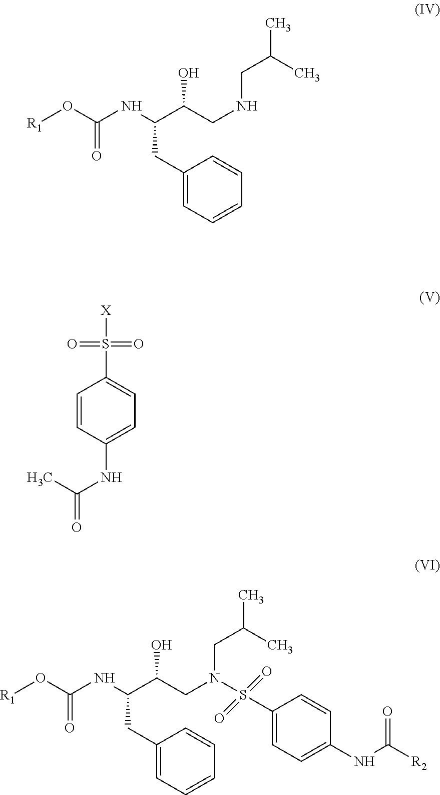
wherein R₁ is methyl, ethyl, t-butyl or benzyl; R₂ is H, methyl, trifluoromethyl or phenyl; X is Cl, Br, I or F; and
b) reacting the compound of formula VI with acid selected from sulfuric acid, hydrobromic acid, nitric acid, methanesulfonic acid or phosphoric acid to give a compound of formula VII.

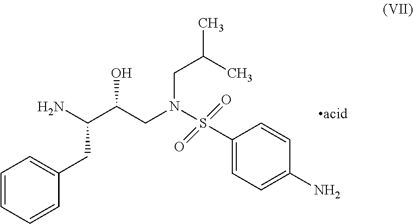 [SMILES: CC(C)CN(C[C@@H](O)[C@@H](N)Cc1ccccc1)S(=O)(=O)c1ccc(N)cc1]

12. The process according to claim 11, wherein step a) is carried out in presence of a base selected from organic base such as triethylamine (TEA), diethylmethylamine, pyridine, 1,8-diazabicyclo[5.4.0]undec-7-ene (DBU), N methyl 2-pyrrolidone (NMP), diisopropyl ethylamine (DIPEA), N,N-dimethyl amino pyridine (DMAP), carbonate bicarbonate or hydride of alkali and alkaline earth metals such as sodium carbonate, sodium bicarbonate, potassium carbonate, potassium bicarbonate, lithium carbonate, cesium carbonate, sodium hydride or mixtures thereof; and a solvent selected from N,N-Dimethylacetamide (DMAc), N.N dimethyl formamide (DMF), NMP, methylene dichloride (MDC), ethylene dichloride (EDC), toluene, ethylacetate (EtOAc), isopropylacetate (iPrOAc), acetone, methylethyl ketone (MEK), tetrahydrofuran (THF), acetonitrile (ACN) or mixture thereof.

13. A compound of formula (VII) wherein acid is sulfuric acid having following formula

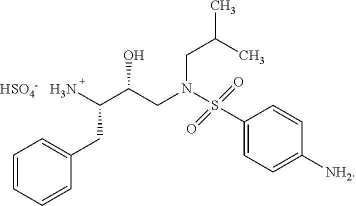 [SMILES: "HSO4-" ; CC(C)CN(C[C@@H](O)[C@@H](N)Cc1ccccc1)S(=O)(=O)c1ccc(N)cc1]

14. A process for preparation of compound of formula (VII) which comprises reacting the compound of formula VI with acid selected from sulfuric acid, hydrobromic acid, nitric acid, methanesulfonic acid or phosphoric acid, 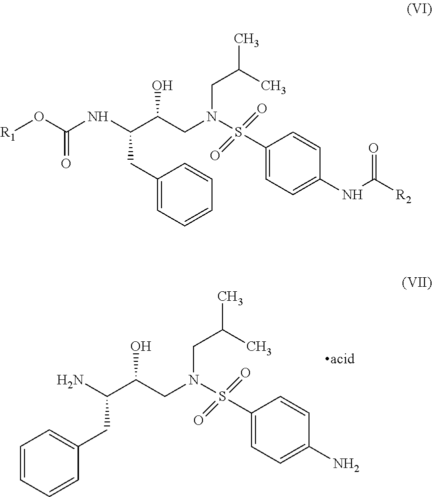
wherein R₁ is methyl, ethyl, t-butyl or benzyl; R₂ is H, methyl, trifluoromethyl or phenyl.

15. (canceled)

16. Darunavir substantially free of bis-impurity of following structure: 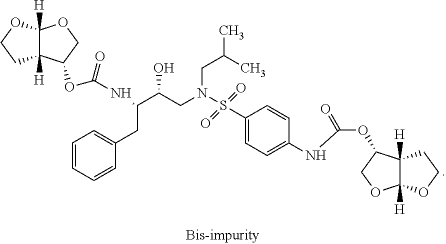 [SMILES: CC(C)CN(C[C@@H](O)[C@H](Cc1ccccc1)NC(=O)O[C@H]1CO[C@H]2OCC[C@H]21)S(=O)(=O)c1ccc(NC(=O)O[C@H]2CO[C@H]3OCC[C@H]32)cc1]
